(19) United States
(12) Patent Application Publication
(10) Pub. No.: US 2010/0286273 A1
(43) Pub. Date: Nov. 11, 2010

(54) FOOD SUPPLEMENT

(76) Inventors: Laura Fretwell, Leicestershire (GB); Sandra McCune, Leicestershire (GB); Janel Fone, Leicestershire (GB); Debby Yates, Melton Mowbray (GB)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 12/066,784

(22) PCT Filed: Sep. 12, 2006
(86) PCT No.: PCT/GB2006/003363

§ 371 Date: Mar. 6, 2009

(30) Foreign Application Priority Data
Sep. 14, 2005 (GB) 0518776.0

(51) Int. Cl. A61K 31/195; A61P 25/00

(52) U.S. Cl. 514/561

(57) ABSTRACT

The present invention relates to the use of leucine, isoleucine and valine in the manufacture of a food supplement for improving the cognitive ability of a companion animal, a pet food supplement comprising leucine, isoleucine and valine and a method of improving the cognitive ability of an animal especially a companion animal.

The present invention relates to the use of leucine, isoleucine and valine in the manufacture of a food supplement for improving the cognitive ability of a companion animal, a pet food supplement comprising leucine, isoleucine and valine and a method of improving the cognitive ability of an animal especially a companion animal.

The interaction between humans and animals has developed to the extent that humans rely on animals to carry out a number of vital functions. The role of dogs in particular as guide dogs for the blind, hearing dogs for the deaf, in tracking, in detecting materials such as drugs and explosives, etc is important to society.

The ability of an animal to carry out a vital or required function or to perform tasks for the pleasure of the animal and the owner or trainer depends on the cognitive ability and/or function of an animal. The improvement in such cognitive ability will provide numerous benefits for the animal and its owner.

The inventors have identified that the branched chain amino acids leucine, isoleucine and valine can be used to improve the cognitive ability and improve the mental well being of a companion animal, especially a dog.

The first aspect of the invention therefore provides the use of leucine, isoleucine and valine in the manufacture of a food supplement for improving the cognitive ability of a companion animal.

The leucine, isoleucine and valine may be provided as free amino acids, preferably in crystalline form or as a peptide rich in leucine, isoleucine and valine or leucine, isoleucine and valine rich extract. Suitable peptides rich in the leucine, isoleucine and valine include dipeptides, tripeptides, tetrapeptides, pentapeptides, hexapeptides, longer chain peptides or peptide mixtures. Such peptide mixtures include proteins rich in leucine, isoleucine and valine, hydrolates or fractions thereof. The amino acids may further be provided as salts, N-acyl amino acid derivatives and/or N-alkanoyl derivatives such as N-acetyl L-valine etc. Valine, leucine and isoleucine are preferably provided in the L-form. In a preferred feature, leucine, isoleucine and valine are provided with a carbohydrate source.

The food supplement of the first aspect is provided for companion animals (any pet animal) including domestic animals such as cats, dogs, horses etc. The food supplement increases and/or sustains the cognitive ability of a companion aminal. In particular, the food supplement increases and/or sustains the ability of an companion animal to acquire and/or apply knowledge. More particularly the food supplement increases and/or sustains perception, intuition and/or reasoning in a companion animal. The food supplement optimises the mental well being of the animal. The food supplement of the first aspect provides a particular benefit to dogs, in particular for assistance, show, agility, performance, rescue, working and obedience dogs. The use of the food supplement to improve cognitive function of such a dog will provide numerous benefits including improved performance, more efficient performance, improved interaction with the owner or handler, etc. The dog for the present invention can be a working dog for example a sheep dog, a rescue dog, a dog involved in the detection of materials such as drugs or explosives, a tracking dog, a guide dog, a hearing dog for the deaf etc. Alternatively, the dog may be a domestic pet. The food supplement of the first aspect can be used to improve the cognitive ability of an companion animal which is presented in a show especially to improve agility, obedience and/or performance of a companion animal.

The improvement in cognitive ability results in an improvement in observed physical performance. The food supplement of the first aspect can therefore be used to improve and/or sustain both mental and physical performance.

The food supplement can be provided to a companion animal on a regular basis, for example one, two or three times a day. Such regular administration to an animal can allow a sustained improvement in the cognitive ability of an animal and may be useful for an animal such as a guide dog where consistent cognitive ability is required. Alternatively the food supplement can be provided when a particular improvement in cognitive ability is required, for example when an animal has to solve a problem, prior to a period of intense or high cognitive activity such as a show or a rescue, to improve the animal's performance during a period of mental activity or to give the animal extra energy and/or mental ability during a particularly tiring or demanding activity such as during a rescue.

Preferably, the supplement can be provided to a senior animal (aged 8 years and above). A senior animal, e.g. a dog, may, in particular, benefit from the present invention.

The food supplement of the first aspect can be a powder, biscuit, snack bar, sweet product, treat, kibble, sauce, topping, pocket or tablet that can be administered with or without an additional foodstuff.

Where the food supplement is administered with an additional foodstuff, the food supplement can be administered sequentially, simultaneously or separately. The food supplement may be mixed with the foodstuff. The mixing may occur when the foodstuff is prepared or packaged or may occur when the foodstuff is provided to the animal. The leucine, isoleucine and valine may be mixed together before adding to any other components or may be added separately. If separately, they may be added at the same time or different times. Alternatively the food supplement can be sprinkled or poured over the foodstuff (i.e. provided as a topping to the foodstuff) or served separately. Alternatively, the food supplement can be added to a liquid provided for drinking such as water or milk or to a liquid foodstuff (for example, an aqueous or oil-based drink).

The formulation of the food supplement is not limiting. The drink, snack bar or other foodstuff may allow for easy transport, administration and monitoring of the precise supplement given. The foodstuff may also be provided in pellet form. In all cases the leucine, isoleucine and valine supplement may be in any form, such as in the form of a liquid or a solid, for example, a powder.

In a particularly preferred feature of the first aspect, the food supplement is provided as a snack or treat which can be supplied to the companion animal, such as a dog, just before an increase in cognitive performance or ability is required. In particular, the snack bar may contain one or more sources of energy to improve the mental and physical performance of the animal and/or to supply the mental and physical energy requirements of the animal. When the foodstuff is in the form of a snack bar, the leucine, isoleucine and valine may be admixed with the components or applied as a topping.

For the purposes of this invention, the term food supplement also encompasses a foodstuff comprising leucine, isoleucine and valine. The foodstuff can be a standard food product as well as a pet food snack. The foodstuff is preferably a cooked product. It may incorporate meat or animal derived material (such as beef, chicken, turkey, lamb, fish, blood plasma, marrow bone etc or one or more thereof). The product alternatively may be meat free (preferably including a meat substitute such as soya, maize gluten or a soya product) in order to provide a protein source. The product may contain additional protein sources such as soya protein concentrate, milk proteins, gluten etc. The product may also contain a starch source such as one or more grains (e.g. corn, rice, oats, barley etc), or may be starch free. It may include a gelatinised starch matrix.

The foodstuff of the invention is preferably a complete and balanced food or is preferably used in combination with a complete and balanced food (for example, as described in National Research Council, 1985, Nutritional Requirements for Dogs, National Academy Press, Washington D.C. or Association of American Feed Control Officials, Official Publication 1996). A complete and balanced diet includes a high quality commercial food. A high quality commercial food can be defined as a diet manufactured to the nutrient recommendations of the National Research Council, 1985 (supra), wherein the digestibility of key nutrients is 80% or more.

The food supplement is preferably packaged. In this way, the consumer is able to identify, from the packaging, the ingredients in the food supplement and confirm that it is suitable for the particular pet in question. The packaging may be metal (usually in the form of a tin or flexifoil), plastic (usually in the form of a pouch or bottle), paper or card. The amount of moisture in any product may influence the type of packaging, which can be used or is required.

In particular, the food supplement of the present invention is food for administration to a companion animal before any cognitive task.

The second aspect of the invention provides a method for improving the cognitive ability of a companion animal, said method comprising administering a food supplement as defined in the first aspect of the invention to a companion animal in need thereof.

The third aspect of the invention provides a pet food supplement comprising leucine, isoleucine and valine. The food supplement can be a powder, biscuit, snack bar, sweet product, treat, kibble, sauce, topping, pocket or tablet that can be administered with or without an additional foodstuff. For the purposes of the third aspect, the term food supplement preferably does not relate to a drink or a food supplement which is added to a liquid and administered to the animal as a drink.

The third aspect of the invention particularly relates to a snack bar comprising leucine, isoleucine and valine.

A fourth aspect of the invention relates to a food supplement as defined in the third aspect of the invention for use in improving or sustaining cognitive ability.

A fifth aspect of the invention relates to a method of manufacturing the food supplement of the first to fourth aspects. The food supplement or foodstuff can be made according to any method known in the art such as in Waltham Book of Dog and Cat Nutrition, Ed. ATB Edney, Chapter by A. Rainbird, entitled “A Balanced Diet” in pages 57 to 74 Pergamon Press Oxford.

In particular, the components of the food supplement can be added together or separately at any time during the processing. They may all be added together at the same time, or individually, in any particular order. Other ingredients of the food supplement and/or foodstuff may be added at any time during the processing. Preferably, two or more ingredients of the food supplement are mixed together and then ground together. The moisture and temperature of the ground particles can be manipulated prior to any further processing step. The components may be added before or after any heating or cooking step. The processing may include shaping and/or packaging of the product. In a preferred feature of the fifth aspect, the product is shaped by extrusion to form pellets or kibbles. Extrusion preferably occurs at a pressure of 20-1000 psig and a temperature of 90-165° C.

The components of the food supplement (valine, leucine and isoleucine) may be mixed with the other components of the food supplement or can be added to the completed food supplement. In a preferred feature of the invention, one or more of the components (for example, valine, leucine and/or isoleucine) is coated or sprayed on to the surface of the food supplement. Alternatively, one or more of the components are admixed, with one or more other components of the food supplement. The final water content of the food supplement can be manipulated using a cooler apparatus.

All features of each of the aspects of the invention apply to all other aspects mutatis mutandis.

The invention will now be illustrated by reference to the following non-limiting examples.

### Methods

A study was prepared to investigate the effect of leucine, isoleucine and valine on cognitive function in active dogs.

The study comprised a total of 24 dogs, of which 12 were in the young age-group (aged 1-3 years old) and the other 12 in the senior age-group (aged 8 years and above). Six dogs in each group were given a leucine, isoleucine and valine treat (in the form of a rice cake) on the day of the trial and the other six in each group acted as a control and received an unsupplemented rice-cake.

The 24 dogs selected for the study completed a seven-week training period to complete a standard agility course.

### Branched-Chain Amino Acid Supplementation

Valine, leucine and isoleucine were provided to the dogs at a ratio of 40% valine, 35% leucine and 25% isoleucine in a 7% carbohydrate solution (7 g/100 g water). The dosage of leucine, isoleucine and valine was calculated based on a 30 kg Labrador retriever. The branched chain amino acid mix was used to coat a low protein, high carbohydrate rice-cake. This coating was done evenly so that the rice-cake could be split into smaller portions for lighter dogs and fed according to body weight.

Control rice cakes were impregnated with a baste to improve palatability while treated rice cakes had the leucine, isoleucine and valine added to the baste.

On the day of the test the rice-cake was fed as a proportion of bodyweight, for example, a 30 kg Labrador was given one rice-cake whereas a 15 kg English Springer Spaniel was given half a rice-cake.

The rice cakes were analysed for moisture, protein, fat and ash, sugars and amino acids. The complete set of results is shown in Table 1.

TABLE 1
Analysis of supplemented and unsupplemented rice cakes

| | Supplemented Rice | Unsupplemented Rice |
|---|---|---|
| Analysis | Cake (%) | Cake (%) |
| Moisture | 17.7 | 14.8 |
| Protein | 12.8 | 8.6 |
| Fat | 3.6 | 5.7 |
| Ash | 4.8 | 4.8 |
| Valine | 4.73 | 0.40 |
| Leucine | 3.89 | 0.55 |
| Isoleucine | 2.88 | 0.27 |

From these results a Predicted Metabolisable Energy Intake (PME) was calculated for both the supplemented and unsupplemented rice cakes as set out below.

### Calculation of PME of Supplemented and Unsupplemented Rice-Cakes

PME (kcal/100 g)=Protein %×3.5+Fat %×8.5+NFE %*×3.5

*NFE %=100−Protein %−Fat %−Moisture %−Ash %−Crude Fibre % (if known)

### Therefore;

### Supplemented Rice Cake

$$\begin{matrix}PME = 12.8 \times 3.5 + 3.6 \times 8.5 + 61.1 \times 3.5 \\ = 44.8 + 30.6 + 213.85 \\ = 289.25 kcal \text{/} 100 g\end{matrix}$$

### Unsupplemented Rice Cake

$$\begin{matrix}PME = 8.6 \times 3.5 + 5.7 \times 8.5 + 66.1 \times 3.5 \\ = 30.1 + 48.45 + 231.35 \\ = 309.9 kcal \text{/} 100 g\end{matrix}$$

The PME of the supplemented rice cake was calculated as 289.25 kcal/100 g; and the unsupplemented as 309.9 kcal/100 g. This is approximately 52 kcal per supplemented cake and 43 kcal per unsupplemented cake. Each supplemented cake contained 0.85 g valine, 0.70 g leucine, 0.52 g isoleucine.

### Dog Selection

Twenty-four dogs were categorised into two age groups: Young (aged between 1.5 and 3.5 years) and Senior (over 8 years of age) (12 senior and 12 young). The dogs were selected based on matching age and breed across two groups (supplemented and unsupplemented).

### Agility Training

Each dog received agility training on a daily basis for 7 weeks. Five different types of agility equipment were selected to make up the standard course:
1. Hurdle
2. A-Frame
3. Rigid Tunnel
4. Tyre
5. Collapsible Tunnel

The standard course was arranged in the following order.
1. Hurdle
2. Hurdle
3. A-Frame
4. Left Turn
5. Rigid Tunnel
6. Tyre
7. Right Turn
8. Hurdle
9. Collapsible Tunnel

A training schedule was arranged which allocated blocks of the course for the dogs to learn each week.

### Test Procedures

At the end of the seven-week training period all the dogs had reached a minimum level of performance. This level was decided by the trainer's opinion of their dog's performance in the last two weeks of training and an assessment by an adjudicator of the dog's performance on the last day of training.

A day was set in each of the three weeks following training for individual dog assessment and the final date for the test.

On the test day the dogs were allocated a test time. The branched chain amino acids were provided to the supplemented group 153.8±3.8 minutes before the start of the test. The dog trainers were blind as to which rice-cake their dog received.

Each dog ran through the standard course three times in succession. Immediately afterwards they were walked to a nearby field (approximately 50 metres) and were encouraged to run through the novel course. The novel course consisted of the same equipment as the standard course but in a different order and different location.

The novel course was arranged in the following order:
1. Tyre
2. Hurdle
3. A-Frame
4. Right Turn
5. Collapsible Tunnel
6. Hurdle
7. Left Turn
8. Hurdle
9. Rigid Tunnel

Measures of performance were recorded.

### Assessment

The dogs were scored on their ability to complete each piece of equipment of the course including the turns. If a dog successfully completed an obstacle they were given a tick; if the dog hesitated, completely missed the obstacle, sniffed around it or were distracted, or didn't complete the component properly they were given a cross and were allowed to start that component again. The dog was allowed five attempts at each component before they were allowed to move on until the next. The time taken to complete the course was also recorded; this was taken from when the dog was released from its starting position until it completed the last component.

### Statistical Analysis

TABLE 3
Dog allocation

| Age Group | Supplemented | Unsupplemented | N |
|---|---|---|---|
| Young | 1 Cocker Spaniel | 1 Cocker Spaniel | 12 |
| | 5 Labrador Retrievers | 5 Labrador Retrievers | |
| Senior | 1 English Springer | 1 English Springer | 12 |
| | Spaniel | Spaniel | |
| | 1 Golden Retriever | 2 Golden Retrievers | |
| | 4 Labrador Retrievers | 3 Labrador Retrievers | |
| N | 12 | 12 | |

The number of errors and time taken to complete Round 2 of the standard course and Round 4 (the novel course) are the data used for this statistical analysis.

Supplementation effect on performance in Rounds 2 and 4 was calculated by comparing the number of errors made using Multifactor ANOVA.

In order to assess individual improvement between Round 2 and Round 4 an improvement co-efficient was calculated for each dog. The calculation for this was:

Errors (or Time) % Difference=((Round 4 Errors (or Time)−Round 2 Errors (or Time))/Round 2 Errors (or Time))*100

Multifactor ANOVA was also used to compare performance between Round 2 and Round 4 for supplementation status, breed and age.

Supplementation effect on Round 4 performance was calculated by comparing the number of errors made using Multifactor ANOVA.

### Results

There was no effect of supplementation on baseline performance in Round 2 (mean no. of errors=2.58±2.71 for supplemented and 2.33±2.50 for unsupplemented).

Similarly, there was no significant difference (p>0.2) between supplemented & unsupplemented groups in the total number of errors made in Round 4 (mean no. of errors=3.92±3.00 for supplemented and 5.33±2.57 for unsupplemented).

The improvement coefficient was used to measure the difference in individual dog's baseline performance in Round 2 and their performance in Round 4 (mean error percentage difference). All but two dogs showed an increase in errors between Round 2 and Round 4.

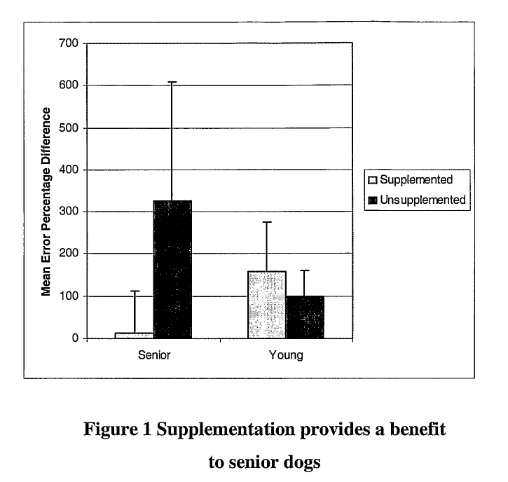

A significant interaction (p<0.02) was found for supplementation and age (mean error percentage difference was senior supplemented 13.33±99.33, senior unsupplemented 325.00±282.40, young supplemented 158.33±115.83, young unsupplemented 98.33±62.98). Supplementation appears has more of an impact on senior dogs than younger dogs (FIG. 1). The error bars on the graph of FIG. 1 refer to Standard Deviations.

### Discussion

Many working dogs face the challenge of maintaining performance over time. The study design was considered to provide a high level of physical and cognitive challenge but without compromising the dogs' welfare. Senior dogs made less total errors than young dogs on Round 4 (the unfamiliar course).

All but two dogs in this study showed an increase in errors as they progressed from Round 2 (the familiar course) to Round 4. This loss of performance was greatly reduced for senior dogs if they were supplemented. This study shows that supplementation with leucine, isoleucine and valine provides a cognitive benefit for active dogs, particularly if they are senior.

### CONCLUSIONS

These preliminary data show that supplementation with leucine, isoleucine and valine improves the ability of senior dogs, in particular, to sustain cognitive performance during exercise, which may impact on overall performance.

1.-16. (canceled)

17. A method for improving the cognitive ability of a companion animal comprising:
administering to a companion animal a pet food supplement comprising leucine, isoleucine, and valine.

18. The method of claim 17, wherein the food supplement is a powder, biscuit, snack bar, sweet product, treat, kibble, sauce, toping, pocket or tablet.

19. A pet food supplement comprising:
leucine, isoleucine, and valine;
wherein the pet food supplement is a powder, biscuit, snack bar, sweet product, treat, kibble, sauce, topping, pocket or tablet; and
wherein the pet food supplement improves or sustains the cognitive ability of a companion animal.

20. The pet food supplement of claim 19, wherein the leucine, isoleucine, and valine are provided as free amino acids, a peptide rich in one or more of the leucine, isoleucine, and valine, or a leucine, isoleucine, and valine rich extracts.

21. The pet food supplement of claim 19, wherein the food supplement is suitable for a dog.

22. The pet food supplement of claim 19, wherein the food supplement is suitable for a senior dog.

23. The pet food supplement of claim 19, wherein the food supplement improves and/or sustains mental and physical performance.

24. The pet food supplement of claim 19, further comprising a meat or animal derived material, a meat substitute, a supplemental protein source, a starch, or a combination thereof.

25. The pet food supplement of claim 24, wherein the meat or animal derived material is beef, chicken, turkey, lamb, fish, blood plasma, bone marrow, or a combination thereof.

26. The pet food supplement of claim 24, wherein the meat substitute is soya, maize gluten, or a soya product.

27. The pet food supplement of claim 24, wherein the supplemental protein source is soya protein concentrate, milk protein, gluten, or a combination thereof.

28. The pet food supplement of claim 24, wherein the starch is corn, rice, oats, barley, or a combination thereof.

29. The pet food supplement of claim 24, wherein the starch is gelatinized.

30. The pet food supplement of claim 17, wherein the food supplement is cooked.

31. The pet food supplement of claim 17, wherein at least about 80% of the key nutrients of the pet food supplement are digestible.

32. A method of manufacturing a pet food supplement comprising:
combining leucine, isoleucine, and valine with a component of a food supplement.

33. The method of claim 32, wherein the leucine, isoleucine, and valine are combined with the component of the food supplement prior to cooking.

34. The method of claim 32, wherein the leucine, isoleucine, and valine are combined with the food component in an extruder to form a pellet or kibble.

35. The method of claim 32, wherein the leucine, isoleucine, and valine are provided as free amino acids, a peptide rich in one or more of the leucine, isoleucine, and valine, or a leucine, isoleucine, and valine rich extract.

36. The method of claim 32, wherein the food component is a meat or animal derived material, a meat substitute, a supplemental protein source, a starch, or a combination thereof.
